(19) United States
(12) Patent Application Publication
(10) Pub. No.: US 2020/0030191 A1
(43) Pub. Date: Jan. 30, 2020

(54) Adjustable Feeding Tube Holder

(71) Applicants: Joseph Santillan, Rosemead, CA (US); Martha Santillan, Rosemead, CA (US)

(72) Inventors: Joseph Santillan, Rosemead, CA (US); Martha Santillan, Rosemead, CA (US)

(21) Appl. No.: 16/378,797

(22) Filed: Apr. 9, 2019

Related U.S. Application Data
(60) Provisional application No. 62/703,028, filed on Jul. 25, 2018.

(51) Int. Cl. A61J 15/00; F16B 2/12; F16B 2/18; A61G 5/10; F16B 2/00; A61G 7/05

(52) U.S. Cl. CPC A61J 15/0053; F16B 2/12; F16B 2/185; A61M 2025/024; F16B 2/005; A61G 7/0503; A61G 5/10

(57) ABSTRACT

An adjustable feeding tube holder. The adjustable feeding tube holder includes an elongated rod member having a clamp head affixed to one end and a connection member affixed to the second end. A second clamp member is disposed along, and slidably engaged to, the elongated rod member such that the second clamp member can be manually positioned along the rod member via a trigger handle. The clamp head and the second clamp member are configured to removably attached the device to a chose surface such as a wheelchair, bed rail, or table. The first connection member is rotatably connected to the elongated rod. A second connection member having a tube holder on an end thereon is rotatably and slidably connected to the first connection member. The tube holder configured to receive a feeding tube therein.

### CROSS REFERENCE TO RELATED APPLICATIONS

This application claims the benefit of U.S. Provisional Application No. 62/703,028 filed on Jul. 25, 2018. The above identified patent application is herein incorporated by reference in its entirety to provide continuity of disclosure.

### BACKGROUND OF THE INVENTION

The present invention relates to a support stand having adjustable brackets for use in holding objects at a desirable or optimal position as selected by a user. More specifically, the present invention relates to an adjustable feeding tube holder which may be attached to a surface such as a wheelchair or bed rail using a support arm which is pivotally and slidably connected to the upper end of a support rod.

Feeding tubes may be a necessary method for people with severe physical disabilities, illnesses, or similar ailments which may affect their ability to eat regularly. Independently using a feeding tube may be difficult or nearly impossible for someone with physical limitations. The feeding tube and syringe must often be held upright, and people with limited mobility may be unable to grip the items without dropping them. Further, such people may need assistance while eating, which can be messy and frustrating. Accordingly, a device that is configured to enable an individual with limited mobility to easily eat a suitable food therewith is desired.

In light of the devices disclosed in the known art, it is submitted that the present invention substantially diverges in design elements from the known art and consequently it is clear that there is a need in the art for an improvement to existing adjustable feeding tube holders. In this regard, the instant invention substantially fulfills these needs.

### SUMMARY OF THE INVENTION

In view of the foregoing disadvantages inherent in the known types of adjustable support stands now present in the known art, the present invention provides an adjustable feeding tube holder wherein the same can be utilized for providing convenience for the user when providing an attachment for selectively positioning a feeding tube.

It is therefore an object of the present invention to provide a new and improved adjustable feeding tube holder that has all of the advantages of the known art and none of the disadvantages. The present system comprises an elongated rod member having a clamp head affixed to a one end thereon and a first connection member rotatably affixed to a second end thereon, a second clamp member is slidably disposed on the elongated rod member and manually engaged using a trigger handle, a second connection member having a tube holder on an end thereon slidably and rotatably affixed to the first connection member.

It is another object of the present invention to provide an adjustable feeding tube holder wherein the clamp head and the second clamp member have complimentary clamping surfaces thereon with textured patterns for additional grip when removably attached to as surface therebetween.

Another object of the present invention is to provide an adjustable feeding tube holder wherein the trigger handle of the second clamping member comprises a spring-biased trigger which engages the elongated rod when the trigger is released and allows slidable adjustment along the elongated rod member when engaged.

Yet another object of the present invention is to provide an adjustable feeding tube holder wherein the tube holder affixed to an end of the second connection member comprises a tubular channel having an open first end and an open second end and is configured to receive the body of a feeding tube therein through either the first end or the second end.

Still another object of the present invention is to provide an adjustable feeding tube holder wherein fasteners such as thumb screws disposed on the first and second connection members allow a user to selectively affix the members at desired lengths and angles relative to the elongated rod member.

Other objects, features and advantages of the present invention will become apparent from the following detailed description taken in conjunction with the accompanying drawings.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of an embodiment of the adjustable feeding tube holder positioned at varying angles.

FIG. 2A shows a perspective view of an embodiment of the adjustable feeding tube holder with second connection member in an extended configuration.

FIG. 2B shows a perspective view of an embodiment of the adjustable feeding tube holder with second connection member in a retracted configuration.

FIG. 3 shows a perspective view of an embodiment of the adjustable feeding tube holder showing the trigger mechanism of the second clamp member.

FIG. 4 shows a perspective view of an embodiment of the adjustable feeding tube holder removably affixed to a wheelchair with feeding tube installed.

FIG. 5 shows a perspective view of an embodiment of the adjustable feeding tube holder retracted and in a container for storage.

### DETAILED DESCRIPTION OF THE INVENTION

Reference is made herein to the attached drawings. Like reference numerals are used throughout the drawings to depict like or similar elements of the adjustable feeding tube holder. For the purposes of presenting a brief and clear description of the present invention, the preferred embodiment will be discussed as used for providing a chair or bedrail attachment for removable securing a feeding tube at the desired angle. The figures are intended for representative purposes only and should not be considered to be limiting in any respect.

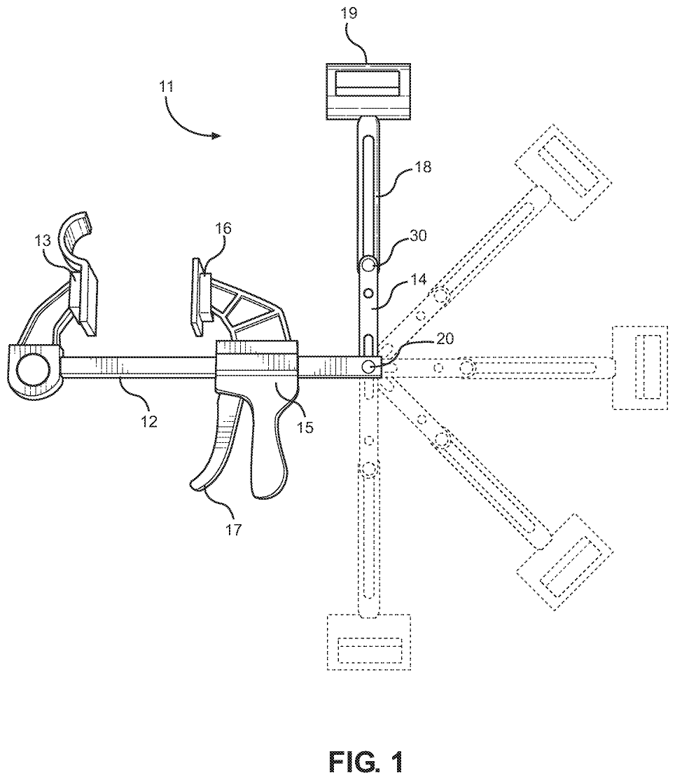

Referring now to FIG. 1, there is shown a perspective view of an embodiment of the adjustable feeding tube holder positioned at varying angles. The adjustable feeding tube holder 11 comprises an elongated rod member 12 having a first end and a second end. The elongated rod member 12 is made of a rigid material and is uniform in diameter. A clamp head 13 is affixed to the first end of the elongated rod member while a first connection member 14 is rotatably affixed to the second end thereon. A second clamp member 15 is disposed on the elongated rod member 12 between the first clamp head 13 and the first connection member 14. The second clamp member 15 comprises an opposing clamp head 16 and trigger handle 17 having a channel therethrough which is adapted to receive the elongated rod member 12 therein in slidable engagement.

A second connection member 18 is slidably and rotatably connected to the first connection member 14. A slot disposed in the second connection member allows a fastener to be received therethrough and affixed to an end of the first connection member 14. This fastener may be loosened to allow the second connection member 18 to freely slide to selectively adjust the length relative to the first connection member 14, or it may be tightened to hold the second connection member 18 in place at a desired position via friction fit. In the illustrated embodiment, the fastener comprises a thumbscrew which is received within a threaded female adaption within the first connection member 14. A tube holder 19 is disposed on an end of the second connection member 18 wherein the tube holder 19 comprises a tubular channel having an open first end and an open second end configured to receive and frictionally engage the body of a feeding tube or similar apparatus therein. The tube holder 19 is adapted to receive a feeding tube or similar apparatus therein through either the first open end or second open end such that either end may be used interchangeably. In the exemplary embodiment, the tube holder 19 includes apertures within the tubular channel which allows for a user to view the feeding tube itself to gauge the volume held within. Further embodiments may include a tube holder which comprise a collar clamp or other adjustable clasp.

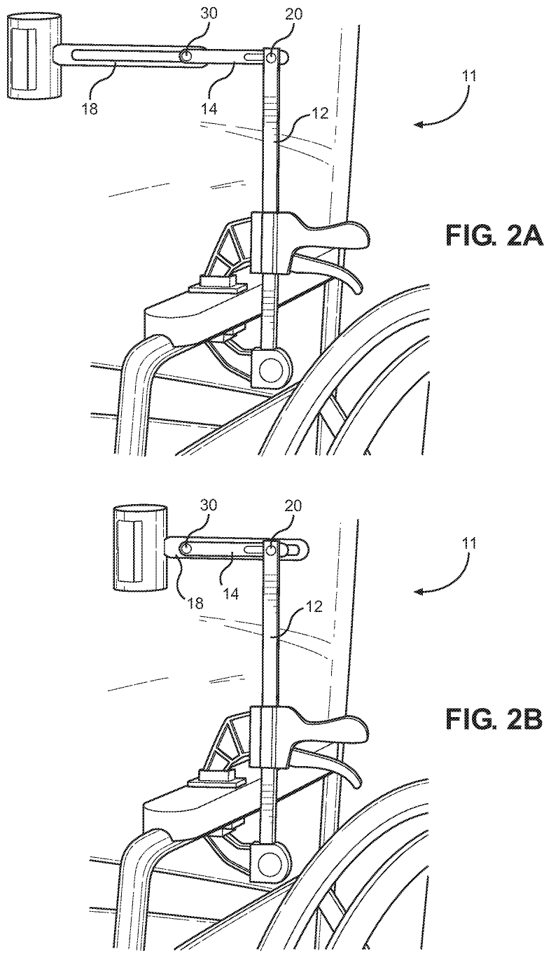

Referring now to FIG. 2A and FIG. 2B, there is shown a perspective view of an embodiment of the adjustable feeding tube holder with second connection member in an extended and retracted configuration, respectively. The first and second connection members 14 and 18 are configured to be selectively affixed at specific angles and lengths as desired by a user. The first connection member 14 is rotatably affixed to an end of the elongated rod 12 while the second connection member 18 is rotatably and slidably connected to the first connection member 14. The second connection member 18 having a channel therein which allows a user to selectively adjust the length of the second connection member 18 with respect to the first connection member 18.

A pair of adjustable fasteners 20 and 30 connect the second connection member 18 to the first connection member 14 and the first connection member 14 to the elongated rod 12. The first adjustable fastener 20 affixes the first connection member 14 to the elongated rod 12 and allows a user to selectively tighten the fastener 20 so as to keep it in a specific angle in relation to the elongated rod 12. In the illustrated embodiment, the adjustable fastener 20 comprises thumb screws which may be tightened within a threaded female adaption disposed on the elongated rod 12 to provide a frictional fit at a certain angle or loosened to allow the first connection member 14 to rotate about the end of the elongated rod 12. Alternate embodiments of the adjustable feeding tube holder 11 may include a plurality of connection members with the exemplary embodiment comprising a first connection member 14 and a second connection member 18.

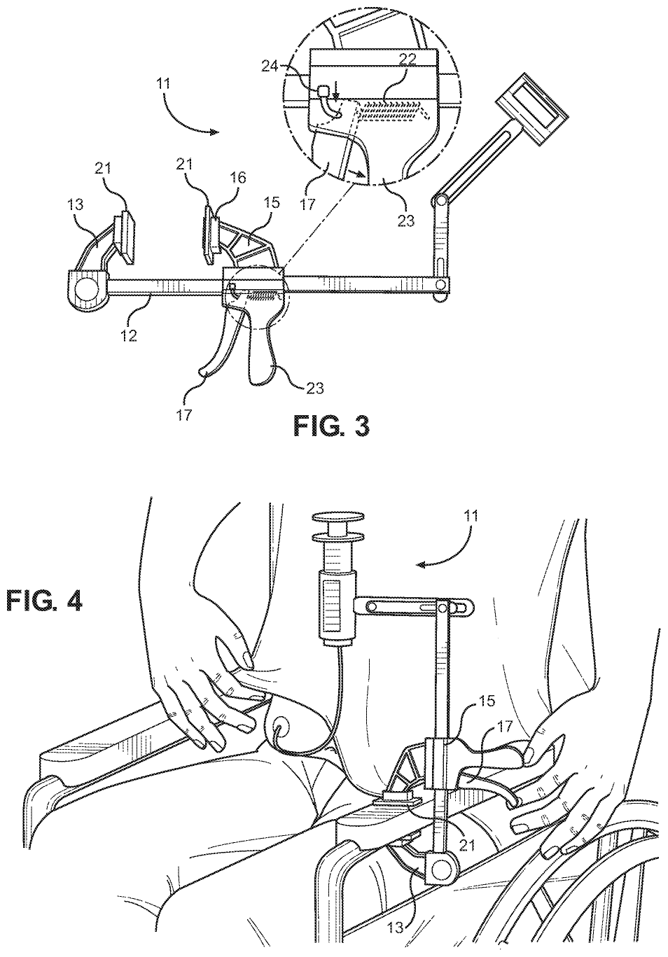

Referring now to FIG. 3 and FIG. 4, there is shown a perspective view of an embodiment of the adjustable feeding tube holder showing the trigger mechanism of the second clamp member and a perspective view of an embodiment of the adjustable feeding tube holder removably affixed to a wheelchair with feeding tube installed, respectively. The adjustable feeding tube holder 11 is configured to removably secure to a surface via friction fit between the clamp head 13 and second clamp member 15. The second clamp member 15 comprises an opposing clamp arm 16 opposite that of the clamp head 13 which is slidably adjustably along the elongated rod 12 via the trigger handle 17. The clamp head 13 and the opposing clamp arm 16 have clamping surfaces 21 which directly oppose each other and have identical dimensions. The trigger handle 17 allows a user to manipulate the second clamp member 15 along the elongated rod 12 to removably attach the device 11 to an anchoring surface. The trigger handle 17 allows a user to lock the second clamp member 15 in place via friction fit provided by a spring-bias against the trigger handle 17. A spring 22 affixed to the trigger handle 17 and grip portion 23 is biased to keep the trigger handle 17 in an opened position. An opened position is defined when the trigger handle 17 is separated from the grip portion 22. When in an opened position, a stopper 24 engages the elongated rod 12 to prevent the second clamp member 15 from moving along the elongated rod 12. When the trigger handle 17 is depressed into a closed position, such that the trigger handle 17 is pulled toward the grip portion 23, the spring 22 is compressed and the stopper 24 is disengaged from the elongated rod 12. When in a closed position, the second clamp member 15 may be selectively positioned along the elongated rod member 12.

In practice, the adjustable feeding tube holder 11 is attached to a surface such as a wheelchair, bed rail, or table to provide a portable stand for those who require feeding tubes or similar devices to provide greater flexibility of action and mobility. Having a highly portable adjustable feeding tube holder 11 gives a user more ease off mind and allows for additional travel options without having to factor in bulky wheeled stands. The clamping surface 21 of both the clamp head 13 and the opposing clamp arm 16 include a textured pattern thereon. The textured pattern provides increased frictional engagement with the chosen anchoring surface to increase stability and prevent lateral movement while also decreasing the likelihood that the adjustable feeding tube holder 11 will become dislodged. In the illustrated embodiment, the adjustable feeding tube holder 11 includes a layer of rubber over the clamping surfaces 21 of the clamp head 13 and the second clamp member 15 to increase the frictional engagement between the clamping surfaces 21 and the anchoring surface. The trigger handle 17 allows for a user to disengage from an anchoring surface and removably secure the device 11 to a different surface area quickly for easy transitions while on the move.

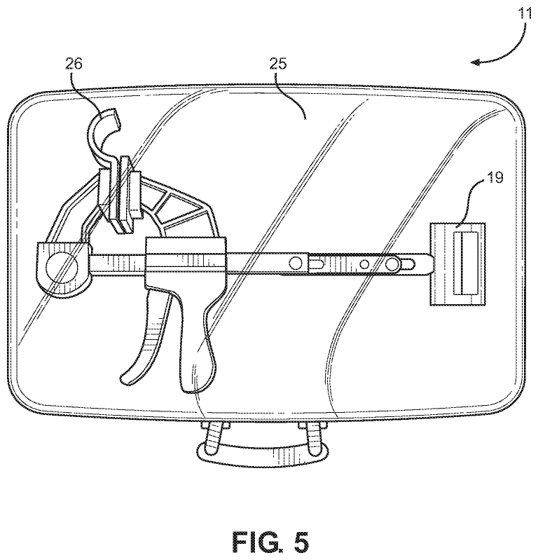

Referring now to FIG. 5, there is shown a perspective view of an embodiment of the adjustable feeding tube holder retracted and in a container for storage. In alternate embodiments of the adjustable feeding tube holder 11, the tube holder 19 may be adapted to receive items having different medical or hygiene purposes. These may include IV fluid containers as well as colostomy or catheter receptacles. The clamp head 13 includes a semi-circular bar attachment 26 in some embodiments, which allows the device 11 to better fastener to the underside of a wheelchair frame. The adjustable feeding tube holder 11 can be stored within a travel case 25 for easy storage and transportation between uses.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

We claim:

1) An adjustable feeding tube holder, comprising:
An elongated rod member having a first end and a second end wherein a clamp head is affixed to the first end thereon and a first connection member s rotatably connected to the second end thereof;
a second clamp member having a channel therein adapted to receive the elongated rod member therethrough, the clamp member being slidably adjustable along the elongated rod member;
a second connection member having a tube holder affixed to an end thereon, wherein the second connection member is slidably and rotatably connected to the first connection member.

2) The adjustable feeding tube holder of claim 1, further comprising a trigger activated fastening system on the second clamp member wherein the member may be slidably positioned along the elongated rod member when then triggeris activated and removably affixed along the rod member when the trigger is deactivated.

3) The adjustable feeding tube holder of claim 2, wherein the trigger activated fastening system includes a spring-biased trigger which creates a friction fit with the elongated rod member when the trigger is deactivated

4) The adjustable feeding tube holder of claim 1, wherein the clamp head and the second clamp member have opposing clamping surfaces of identical size and shape.

5) The adjustable feeding tube holder of claim 4, wherein the clamping surfaces of the clamp head and the second clamping member have a textured pattern to provide increased friction when removably attached to a surface.

6) The adjustable feeding tube holder of claim 4, wherein the clamping surfaces of the clamp head and the second clamping member include a layer of friction increasing material.

7) The adjustable feeding tube holder of claim 1, wherein the distance between the first connection member and the second connection member is selectively adjustable.

8) The adjustable feeding tube holder of claim 7, wherein the connection members are selectively adjustable via a fastener disposed through a slot on the second connection member and affixed the first which may be tightened to hold the second connection piece in frictional engagement.

9) The adjustable feeding tube holder of claim 1, further comprising fasteners included on the first connection member and the second connection member which selectively affix the members at the chosen lengths and angles.

10) The adjustable feeding tube holder of claim 9, wherein the fasteners included on the first connection member and the second connection member comprise thumb screws.

11) The adjustable feeding tube holder of claim 1, wherein the tube holder comprises a tubular channel having an open first end and an open second end configured to receive and frictionally engage the body of a feeding tube therein.

12) The adjustable feeding tube holder of claim 11, wherein the tube holder is adapted to receive and frictionally engage the body of a feeding tube through both the open first end and open second end.

13) The adjustable feeding tube holder of claim 1, further comprising a semi-circularbar attachment disposed on the clamp head configured to receive a metal rod therein.
